(19) United States
(12) Patent Application Publication
(10) Pub. No.: US 2026/0308478 A1
(43) Pub. Date: Oct. 8, 2026

(54) PROCESS FOR PREPARING CHEMICAL FORMULATIONS COMPRISING A NEUTRALIZED COMPONENT

(71) Applicant: BASE SE, Ludwigshafen am Rhein (DE)

(72) Inventors: Simon REIFEGERSTE, Ludwigshafen am Rhein (DE); Chee Jian CHAN, Ludwigshafen am Rhein (DE); Daniela ALTVATER, Ludwigshafen am Rhein (DE); Karin EBERIUS, Ludwigshafen am Rhein (DE); Bjoern KAIBEL, Ludwigshafen am Rhein (DE)

(21) Appl. No.: 19/482,813

(22) PCT Filed: May 3, 2024
(86) PCT No.: PCT/EP2024/062189

§ 371 Date: Nov. 10, 2025

(30) Foreign Application Priority Data
May 12, 2023 (EP) 23173016.9

(51) Int. Cl. C11D 1/22; C11D 1/722; C11D 1/835

(52) U.S. Cl. CPC C11D 1/22; C11D 1/722; C11D 1/835

(57) ABSTRACT

The present invention relates to a continuous process for preparing a homogeneous chemical formulation comprising at least water, an ionic surfactant and a co-surfactant. The process comprises the steps of mixing at least one precursor of the ionic surfactant, the co-surfactant and the neutralization agent by at least one mixing device selected from the group consisting of continuous stirred tank reactors, high shear mixers, mixing pumps, static mixers and mixing nozzles, and removing the obtained chemical formulation is through an outlet. The process is suitable for blending surfactant formulations comprising a neutralized component, and particularly useful for neutralized components which form a gel with water if the co-surfactant would not be present.

### BACKGROUND

Chemical formulations prepared by the neutralization of an acid or base have several applications. For example, surfactant compositions prepared via the neutralization of the acid precursor of the surfactant may be used in oil field, such as in enhanced oil recovery methods, or in gas field, or they may be used in liquid or gel detergent compositions.

The manufacturing of these chemical formulations may sometimes have several challenges, as for example: The neutralized form of the acid or base may react with the water present in the composition, forming a highly viscous product, such as gel, and large amounts of heat are generated during the process. This hinders the handling and further processing of the product, such as mixing of the product with additional components and the set-up of an efficient continuous process, among others.

The blending process is critical. Depending on how the blending is carried-out, there may be problems such as phase separation, agglomeration, presence of solid particles. Some of the reasons causing these problems are the low miscibility of the of precursor, e.g. an acid precursor, in the neutralizing agent solution, e.g. alkali aqueous solution; and the high viscosity products formed when the neutralized ionic surfactant reacts with water forming a gel.

The reaction between the precursor and the neutralizing agent, requires particularly good mixing. As neutralization reactions are very fast and therefore, mostly controlled by mixing speed, a very short mixing time and a high degree of homogeneity is highly beneficial.

Undesired side reactions may take place when additional components are present. For example, when a co-surfactant is present, the co-surfactant may react with the neutralizing agent, or the co-surfactant may react with the precursor. These side reactions are undesired and lead to the formation of undesired byproducts, loss of product performance and degradation of the raw materials. Another consequence is that an excess of neutralization agent may be needed to compensate for the undesired side reaction between the neutralization agent and a third component. Several processes to obtain aqueous solutions of in situ-prepared anionic surfactants are known in the art. However, these known processes still present several disadvantages.

Some known processes lead to a composition comprising a low content of surfactant and high content of water. However, larger amounts of water reduce the active content of the final product, lowering performance and increasing transportation cost and equipment size.

Other known processes lead to final aqueous solutions with high viscosities, which are difficult to handle and pump. That is the case when, for example, a co-surfactant, such as an anionic or nonionic surfactant, is not included in the composition. The co-surfactant may help to improve the solubility of the neutralized acid in the aqueous phase. In some known process where a co-surfactant is used, the precursor is in contact with the neutralizing agent before a fully homogeneous mixture between the precursor and the co-surfactant is obtained. In this case, the gel-phase formed between the neutralized ionic surfactant and the water will not be prevented and it will form as an intermediate phase during the process. This process therefore usually requires the use of larger amounts of water and a neutralization loop with a high shear homogenization unit.

Another disadvantage of known process is that they may be based on non-continuous batch-process, i.e. the reaction is carried out in a mixer into which the starting materials are introduced and the intermediate or end product is removed after a certain mixing and/or reaction time. Batch processes are disadvantageous because they require long preparation times, which are associated with increased production costs. There is also the risk of contamination, which is brought in by the fact that the corresponding systems for the batch process must be emptied and filled again, and then repeated as many times as necessary. It is also undesirable that large amounts of air are often introduced into the system, which can lead to product deterioration.

And when the known processes are based on continuous process, they include the disadvantage of using a loop reactor, i.e. a large part of the reaction mixture circulates through the loop reactor, while only a small part of the mixture is continuously discharged. The disadvantage of such loop reactors is that they require very large volumes and powerful pumps, and a satisfactory back-mixing effect can only be achieved by large circulation ratios or by an additional mixing device. In general, processes using a mixing loop are less efficient compared to single pass systems.

Some of the known processes have been disclosed in the following patents or patent applications: U.S. Pat. No. 6,794,347 B2 aims to reduce the viscosity of the aqueous surfactant solution by preparing chemical formulations with low surfactant content and high water content, in particular, chemical formulations comprising a surfactant and about 50 to 90% of water.

U.S. Pat. No. 4,261,917 discloses a method to prepare an aqueous solution or slurry containing from 60 to 75 wt. % of sulfate of higher alcohol or alkylene oxide adduct of said alcohol. However, the Examples show that the chemical formulations obtained by this method have high viscosities, namely between 130 to 180 Poise at 40° C.

EP 0507402 A1 discloses a continuous process using loop reactors for the production of detergents, in which an acid precursor of an anionic surfactant is neutralized with a neutralizing agent in the presence of a nonionic surfactant, preferably an ethoxylated fatty alcohol.

EP 2922945 B1 and WO 2014/079702A1 disclose a continuous process for producing a liquid detergent or cleaning agent, by mixing continuous separate volume flows of at least an acid precursor of an anionic surfactant and a neutralization agent via a dynamic loop mixer, i.e. a closed in-line mixer with recirculation. Examples of EP 2922945 B1 illustrate a continuous process where firstly, the acid precursor, the neutralization agent and water are combined, the obtained mixture optionally passes through a static mixer and afterwards, the co-surfactant, e.g. fatty alcohol sulfate, is added in the dynamic loop mixer. In particular, in example E2, they disclose a process for preparing a chemical formulation comprising the steps of mixing NaOH and water in a static mixer, followed by adding LAS acid in a dynamic liquid mixer, followed by adding fatty alcohol ether sulfate (an anionic cosurfactant), NaCl and phosphoric acid in another static mixer.

WO 2006/069118 A2 discloses a continuous process for the preparation of a surfactant composition, by mixing a surfactant acid precursor with at least a molar equivalent of a neutralizing agent by using one or more static mixers. Additional components may also be present and may be fed into the process at any stage. However, none of the examples in WO 2006/069118 A2 show a process comprising additional components. Furthermore, the examples also do not mention the viscosity of the obtained chemical formulation, the active content of the surfactant in the obtained chemical formulation, and the molar ratio of the acid precursor and neutralization agent used.

WO 2015/138275 A1 discloses a surfactant composition for use in enhanced oil recovery, comprising a neutralization agent, e.g. NaOH solution, acid precursor of an anionic surfactant, i.e. C_{15/17} alkyl benzene sulfonic acid (ABS) and a co-surfactant, i.e. an ammonium salt of tristyrylphenol (TSP)-PO₃₅-EO₂₅ sulfate. WO 2015/138275 A1 discloses, in an example on page 8 (1^{st} and 2^{nd} paragraphs on page 8), a process where NaOH is mixed with the alkoxy sulfate surfactant, mixed until a homogeneous solution is obtained, and then the ABS is added into the NaOH-Sulfate mixture while mixing.

WO 01/79412 A1 discloses, in an example on page 28, a process for preparing a chemical formulation comprising the steps of mixing linear alkylbenzene sulfonic (LAS) acid and a blend of nonionic surfactants in a static mixer of the Sulzer type, followed adding 50% NaOH to neutralize 30-50% of LAS and pumping the mixture into a static inline mixer, then adding sufficient NaOH to complete neutralization and pumping the resulting mixture through a dynamic inline mixer.

WO 2013/092049 A1 discloses, in comparative example A, a process for preparing a chemical formulation comprising the steps of mixing (among others) C12-14 linear alkylbenzene sulphonic acid (LAS acid) and sodium lauryl ether sulphate with 3 moles EO (SLES 3EO), followed by adding NaOH up to a pH of 6.5, thereby neutralizing the LAS acid.

JP-A 64-47755 discloses, in example 1, a process for preparing a chemical formulation comprising the steps of mixing a nonionic surfactant with LAS acid in an iron reactor tank, followed by adding 45 wt. % NaOH for neutralization. Therefore, it was the object of the present invention to provide an improved continuous process for preparing a chemical formulation which comprises a neutralized component, which reduces undesired side reactions of the process, and leads to a final product with good handling properties.

Surprisingly, the Applicant has discovered an inventive continuous process for manufacturing a chemical formulation comprising water, an ionic surfactant, and a co-surfactant, via the neutralization in situ of the precursor of the ionic surfactant, which provides the following advantages:
an efficient continuous method,
smaller investment into special equipment, such as high shear homogenizers and vessels,
the viscosity of any of the mixtures prepared during the process is not higher than the highest viscosity of the individual components,
the undesired side reactions are avoided or minimized,
reduces the excess of the neutralizing agent necessary, in comparison to other manufacturing methods.

### SUMMARY OF THE INVENTION

Accordingly, the present invention relates to a process for the preparation of a chemical formulation comprising at least water, an ionic surfactant and a co-surfactant, via the neutralization in situ of the precursor of the ionic surfactant, wherein the process is continuous and comprises the steps of:
i) mixing at least one precursor of the ionic surfactant, the co-surfactant and the neutralization agent by at least one mixing device selected from the group consisting of continuous stirred tank reactors, high shear mixers, mixing pumps, static mixers and mixing nozzles,
ii) removing the obtained chemical formulation through an outlet.

### BRIEF DESCRIPTION OF THE DRAWINGS

FIG. 1: Schematic representation of one particular embodiment of the present invention

FIG. 2: Schematic representation of particular embodiments of the present invention, wherein a three-way valve, T-piece and a recirculation pipe are used for the precursor and co-surfactant

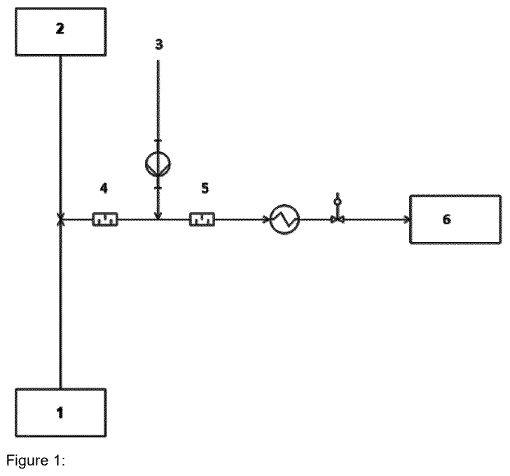

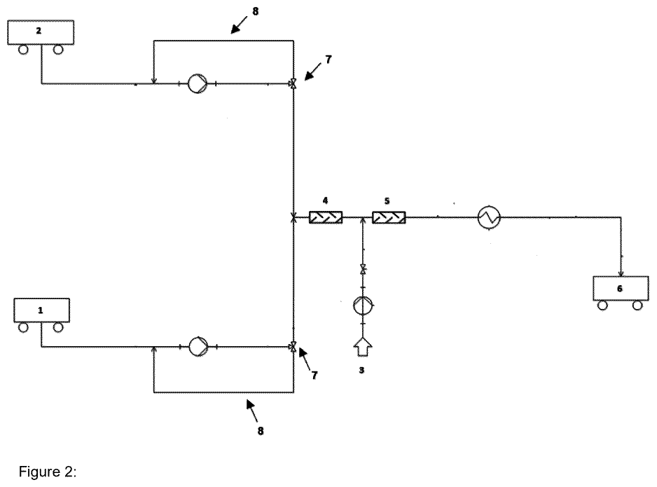

Explanation of the reference numbers use in FIGS. 1 and 2:
(1) source of precursor of the ionic surfactant or neutralization agent,
(2) source of co-surfactant
(3) source of neutralization agent or precursor of the ionic surfactant, whichever is not selected for (1),
(4) first mixing device, preferably a static mixer
(5) second mixing device, preferably a static mixer
(6) outlet for the obtained chemical composition
(7) T-piece and three-way valve connection
(8) recirculation pipe

### DETAILED DESCRIPTION

The specific embodiments, examples and figures herewith disclosed are for illustration purposes and do not limit the scope of the invention.

The continuous process of the present invention comprises the following steps:
i) mixing at least one precursor of the ionic surfactant, the co-surfactant and the neutralization agent by at least one mixing device selected from the group consisting of continuous stirred tank reactors, high shear mixers, mixing pumps, static mixers and mixing nozzles,
ii) removing the obtained chemical formulation through an outlet.

The process is suitable for blending surfactant formulations comprising a neutralized component, and particularly useful for neutralized components which form a gel with water if the co-surfactant would not be present.

More particularly, the disclosed process may be used for the preparation of surfactant blends used in enhanced oil recovery or in liquid or gel detergent compositions, among other applications.

The at least one precursor of the ionic surfactant, the co-surfactant and the neutralization agent may be independently added from their sources into a pipe connected to the mixing device or into the mixing device directly. The term “precursor”, as used through this invention, refers to the acid or basic precursor of the ionic surfactant. The term “source” for any of the components, as used through this invention, is intended to cover any kind of source. In one embodiment, the source may be a tank or an ISO container. Of course, a plurality of sources may be used, for example a plurality of tanks. Preferably, such tanks are mobile, so that they can be easily relocated, for example between manufacturing sites. In certain embodiments of the present invention, the tanks may be tank containers, tank trailers or tank trucks. Basically, the tanks may have any shape and size. In one embodiment, tanks may be cylindrical. The volume of the tanks is not limited. Mobile tanks as mentioned, may have a volume from 1 m³ to 100 m³, for example from 10 m³ to 50 m³. The tanks may also serve as buffer tanks to ensure an uninterrupted supply with the components. When the storage tanks need to be heated, for example to maintain the components pumpable, i.e. with a viscosity lower than 2500 mPas, electrically heated, liquid heated or steam heated iso containers or tanks may be used.

The source of the precursor, the co-surfactant and the neutralization agent may be independently selected.

In another embodiment, the precursor may be manufactured in situ. In this case, the source of the precursor may be understood as the reactor preparing the precursor. The reactor preparing the precursor and the mixing device used in the inventive process may be connected by a pipe. In this embodiment, one or more buffer tanks connected by a pipe may be placed between the reactor preparing the precursor and the mixing device used in the inventive process, to ensure an uninterrupted supply with the precursor.

The term “pipe” as used throughout this invention, encompasses rigid pipes such as pipelines as well as flexible pipes, such as for example hoses or flexible metal pipes. A pipe may of course comprise both, rigid and flexible sections. The diameter of the pipes may be for example from 50 mm to 80 cm. Flows in the system are typically laminar but may also be turbulent.

In one embodiment of the present invention, in step i) the at least one precursor of the ionic surfactant, the co-surfactant and the neutralization agent are added from their sources into the mixing device simultaneously. In this further preferred embodiment, the mixing device is preferably a continuously stirred tank reactor or a high shear mixer. In a different embodiment of the present invention, step i) is divided into four sub-steps, i.e. i-1), i-2), i-3) and i-4), which will be explained in more detail further below. In this further preferred embodiment, the mixing device is preferably a static mixer.

In the present process, the neutralization of the precursor may be partial or full neutralization. In this embodiment, the molar ratio of the acid or base number of the precursor to neutralization agent in in the range of 1:0.1 to 1:2.

In a preferred embodiment, the degree of neutralization of the precursor during the process may be at least 80% wt., more preferably of at least 90% wt., and more preferably substantially all of the precursor is neutralized in the process.

In a further embodiment, the molar ratio of the acid or base number of the precursor to neutralization agent in in the range of 1:1 to 1.1.5, preferably 1:1 to 1:1.1.

In another embodiment, the residence time of the components inside the at least one mixing device may be less than 30 second, preferably less than 15 seconds, more preferably less than 10 seconds.

In a preferred embodiment of the present invention, the pressure inside the mixing device is higher than 200.000 Pa. The time span between the point when the precursor and the co-surfactant get in contact until the point when the homogeneous chemical formulation with the neutralized precursor is obtained is less than 15 minutes, preferably less than 2 minutes, most preferably less than 1 minute.

In a preferred embodiment, the obtained chemical formulation may have a pH value of at least 7, preferably between 8 and 12.

In one embodiment of the present invention, the viscosity of any of the mixtures prepared during the process is not higher than the highest viscosity of the individual components.

The obtained chemical formulation may have a high concentration of surfactants, namely, the chemical formulation may have an active content of at least 80 wt. %, preferably at least 85 wt. %.

After the chemical formulation comprising the ionic surfactant and the co-surfactant exists the mixing device, it shall be cooled down to a stable temperature. For this, heat exchangers or mixing with a cold liquid may be used.

In step ii) of the process, the obtained chemical formulation may be removed by an outlet.

In a preferred embodiment, a heat exchanger is located at or near the outlet of step ii).

In one preferred embodiment, the process does not use a dynamic loop mixer, preferably any loop mixer.

In another preferred embodiment, the process does not use any loop reactor.

Further Description of the Sub-Steps i-1), i-2), i-3) and i-4):

In sub-step i-1), the precursor and co-surfactant may be fed from their sources into a common pipe via a pump. Alternatively, in sub-step i-1), the neutralization agent and the co-surfactant may be fed from their sources into a common pump via a pump.

Any type of pumps may be used for the present process. Preferably, positive displacement pumps or centrifugal pumps are used.

In one embodiment of this invention, recirculation lines may be used to adjust the flows of the precursor and co-surfactant to the required values before feeding these components into the common pipe. For this, a three-way-valve and a T-piece may be used. At the start of the process, the three-way-valve is positioned so that the raw components are fed into the recirculation line, once the required flow values are reached, the position of the three-way-valve is switched, and the raw materials are fed into the common pipe. This embodiment provides the advantage that quality issues during the start-up of the process may be avoided as well as faster start up, shut down and idle procedures can be achieved. This embodiment is illustrated in FIG. 2, where the reference numbers indicate:
(1) source of precursor of the ionic surfactant or neutralization agent,
(2) source of co-surfactant
(3) source of neutralization agent or precursor of the ionic surfactant, whichever is not selected for (1),
(4) first mixing device, preferably a static mixer
(5) second mixing device, preferably a static mixer
(6) outlet for the obtained chemical composition
(7) T-piece and three-way valve connection
(8) recirculation pipe

In sub-step i-2), the mixture of precursor with co-surfactant (or alternatively, the mixture of neutralization agent with co-surfactant) is introduced into at least one first mixing device, selected from a group consisting of continuous stirred tank reactors (CSTR), high shear mixers, mixing pumps, static mixers and mixing nozzles.

In a preferred embodiment, the first mixing device is a static mixer.

High shear mixers are well-known to the skilled person. A high shear mixer is a type of mixing device used in various industries, including pharmaceutical, food, and chemical. It is designed to produce intense mechanical forces that cause liquids and solids to be mixed, dispersed, or emulsified. The mixer typically consists of a rotor and a stator, which create a high velocity flow and impart shear forces of 1000 s⁻¹ or higher on the materials being processed. This high shear action helps break down particles and enhance the uniformity and stability of the mixture. High shear mixers are commonly used in applications where thorough mixing, particle size reduction, or creating stable emulsions is required.

Static mixers are well-known to the skilled person. They have to be capable of operating in a continuous process and of mixing fluids. Suitable static mixers include, for example split-and-recombine type mixers, micro channel mixers and T-Pieces in pipes. The static mixers preferably have a length to diameter ratio of at least 8. It is desired that the inner diameter of the static mixers is similar or equal to the inner diameter of the pipes.

Static mixers are particularly preferred over dynamic mixers for the process of the present invention, as static mixers require lower energy inputs and therefore lead to energy saving and lower operating cost. Static mixers also require lower capital investment, and this is especially true for multi-stage high shear dynamic mixers, which have significantly higher lifetime cost than static mixers used for the process of the present invention.

In one embodiment, the mixture obtained in sub-step i-1) is introduced into the first static mixer directly after the streams of precursor and co-surfactant are unified.

In one embodiment of the present invention, the mixture prepared in step i-2) is substantially homogeneous when exiting the at least one first static mixer. The homogeneity of the mixture can be evaluated by the coefficient of variation of the concentration. In one embodiment, the coefficient of variation of the concentration of the mixture prepared in sub-step i-2) is less than 10%, preferably less than 5%, more preferably less than 1%.

The coefficient of variation of the concentration (CV) can be calculated as follows:

$$CV = \frac{s}{\overline{x}} \times 100 \%$$

where s is the concentration of a randomized sample of the mixture and x is the average concentration of the mixture.

A coefficient of variation of the concentration lower than 10% indicates that the mixture has reached such homogeneity that when taking a randomized sample from the mixture, the concentration of that sample will only deviate from the average concentration of the mixture by 10%. Therefore, the lower the coefficient of variation of concentration, the higher the homogeneity of the mixture.

Achieving a good homogeneity in sub-step i-2) has the advantageous consequence that the viscosity of any of the prepared mixtures during the process is not higher than the highest viscosity of the individual components.

As already stated above, many chemical formulations prepared by the neutralization of an acid or base have the added difficulty that the neutralized form of the acid or base reacts with the water present in the composition, forming a highly viscous product, such as gel. This increases the handling difficulty of the product, the mixing of the product with additional components and the set-up of an efficient continuous process, among others.

This often occurs, for example, in the preparation of surfactant compositions for oilfield or detergent applications via the neutralization of the acid precursor of the surfactant.

In the sub-step i-3) of the process, the mixture obtained in sub-step i-2) is combined with at least one neutralization agent. The neutralization agent reacts with the precursor to form the ionic surfactant. Or alternatively, in the sub-step i-3), the mixture obtained in sub-step i-2) is combined with at least one precursor of the ionic surfactant.

In one embodiment, the time span between the start of sub-steps i-1) and i-3) is less than 10 minutes, preferably less than 2 minutes, especially preferably less than 1 minute. Reducing the time that the non-neutralized precursor is in contact with the co-surfactant is beneficial because these two components can undesirably react, thus, forming undesired byproducts and reducing the efficiency of the process, reducing yield or deteriorate product quality.

For example, when a sulfonic acid derivative is used as precursor and an ammonium salt of a sulfate derivative is used as co-surfactant. The acidic nature of the free sulfonic acid leads to desulfation of the co-surfactant, causing a loss in product performance. A pre-mixture of the sulfate derivative ammonium salt with an alkali as neutralization agent will lead to ion exchange of the ammonium salt with the alkali metal, releasing hazardous ammonia. Therefore, by achieving a substantially homogeneous mixture of the precursor and the co-surfactant under 10 minutes, preferably under 2 minutes, especially preferably under 1 minute, the present process provides all the lower viscosity-advantages of mixing the precursor with the co-surfactant, while avoiding or minimizing the byproduct-disadvantages of the undesired reaction of the precursor with the co-surfactant.

In one of the embodiments of the present invention, the neutralization agent (or alternatively, the precursor of the ionic surfactant) is added to the mixture prepared in sub-step i-2) at a position located after the at least one first mixing device and before the at least one second mixing device.

In the sub-step i-4) of the process, the mixture obtained in sub-step i-3) passes through at least one second mixing device selected from a group consisting of continuous stirred tank reactors, high shear mixers, mixing pumps, static mixers and mixing nozzles.

The characteristics of the second mixing device may be the same or different as for the first mixing device.

In one embodiment, the residence time of the components inside any of the mixing devices, either the first mixing device or second mixing device, may be less than 30 second, preferably less than 15 seconds, more preferably less than 10 seconds.

In a preferred embodiment, the first or second mixing device is a static mixer. In a further preferred embodiment, both the first and second mixing devices are static mixers. The static mixers provide the advantage that homogeneous mixtures may be obtained in a short amount of period, which results in better conversion of the main desired reaction in the process while avoiding or minimizing undesired side-reactions.

Due to the required energy input for the mixing action in the static mixers an increased pressure is present in front of the mixers as compared to behind the mixers when seen in direction of flow. Typically the pressure inside the static mixers is higher than atmospheric pressure. It is particularly preferred that the pressure inside the static mixers is higher than atmospheric pressure, as this plays a role in preventing undesired side-reactions. Namely, this is relevant for relevant for side reactions that are an equilibrium reaction and form side products which have a vapor pressure higher than atmospheric pressure at the process temperature The pressure increase may be created by the flow of the components through the static mixers or by applying pressure with a pump or a gas.

This particular preferred embodiment is illustrated in FIG. 1, where the reference numbers indicate:
(1) source of precursor of the ionic surfactant
(2) source of co-surfactant
(3) source of neutralization agent
(4) first mixing device, preferably a static mixer
(5) second mixing device, preferably a static mixer
(6) outlet for the obtained chemical composition

In another preferred embodiment, the reference numbers of Figure indicate the following:
(1) source of neutralization agent
(2) source of co-surfactant
(3) source of precursor of the ionic surfactant
(4) first mixing device, preferably static mixer
(5) second mixing device, preferably static mixer
(6) outlet for the obtained chemical composition.

If the precursor is an acid, during the neutralization a temperature rise is expected, depending on the acid content in the precursor. Thus, after the homogeneous chemical formulation comprising the ionic surfactant and the co-surfactant exists the second mixing device, it shall be cooled down to a stable temperature. For this, heat exchangers or mixing with a cold liquid may be used.

### Chemical Components

### Abbreviations Used:

PO=propylene oxide
EO=ethylene oxide
TSP=tristyrylphenol
ABS=alkylbenzene sulphonic acid
LABS=linear alkylbenzene sulphonic acid

The term “precursor”, as used through this invention, refers to the precursor of the ionic surfactant.

The ionic surfactant is formed in situ by reaction of an appropriate precursor and a neutralizing agent. Unless specified, the precursor, ionic surfactant and co-surfactant may be either linear or branched molecules. In one embodiment, the ionic surfactant may be an anionic surfactant selected from a group comprising alkylbenzene sulphonates, preferably linear alkylbenzene sulphonates having an alkyl chain length of C₁₀₋₃₂; primary and secondary alkyl sulphonates, preferably C₁₀₋₁₅ primary alkyl sulphates; alkyl ether sulphates; olefin sulphonates, preferably C₁₉₋₂₈ internal olefin sulphonate or C₁₂₋₂₃ alpha-olefin sulfonates; alkyl xylene sulphonates; dialkyl sulphosuccinates; and fatty acid ester sulphonates; and combinations thereof.

Accordingly, in this embodiment, the precursor is the acid form of the at least one selected anionic surfactant. Namely, the precursor may be selected from alkylbenzene sulphonic acid, preferably linear alkylbenzene sulphonic acids (LABS) having an alkyl chain length of C₁₀₋₃₂; primary and secondary alkyl sulphonic acids, preferably C₁₀₋₁₅ primary alkyl sulphonic acids; alkyl ether sulphonic acids; olefin sulphonic acids; alkyl xylene sulphonic acids; dialkyl sulphosuccinic acids; and fatty acid ester sulphonic acids.

In a further preferred embodiment, the ionic surfactant is an alkylbenzene sulphonates, preferably linear alkylbenzene sulphonates having an alkyl chain length of C₁₀₋₃₂, and the precursor is an alkylbenzene sulphonic acid, preferably linear alkylbenzene sulphonic acids (LABS) having an alkyl chain length of C₁₀₋₃₂.

In a further preferred embodiment, the anionic surfactant may have sodium as counterion.

When the ionic surfactant is anionic and the precursor is the acid form of the at least one selected anionic surfactant, the neutralization agent may be a base.

In one embodiment, the neutralizing agent may be a base selected from an aqueous solution of NaOH, KOH,

NH₄OH, Na₂CO₃, NaHCO₃, K₂CO₃, KHCO₃, Ca(OH)₂, Mg(OH)₂, Na acetate, K acetate or NH₄ acetate, preferably an aqueous solution of NaOH.

In a preferred embodiment, the aqueous solution of the neutralizing agent may have an active content of 2 to 98% wt., preferably 20 to 80% wt., more preferably 45 to 60% wt.

In a preferred embodiment of the invention, the precursor is an acid, the neutralization agent is a base, and the first ionic component is anionic.

The co-surfactant may be different from the ionic surfactant and may be selected as ionic or nonionic, preferably anionic or nonionic, more preferably anionic.

Examples of anionic co-surfactants are carboxylates, sulfonates or sulfates.

The carboxylate may be selected from at least one of C₁₂₋₃₂-alkyl PO₂₋₅₀ EO₂₋₁₀₀ carboxylates, C₁₂₋₃₂-alkyl PO₂₋₅₀ carboxylates, C₁₂₋₃₂-alkyl EO₂₋₁₀₀ carboxylates, Tristyrylphenol (TSP) PO₂₋₅₀ EO₂₋₁₀₀ carboxylate, other mono-, di-, and trialkylphenolalkoxycarboxylate, Coco amidopropylbetain, other C₁₂₋₂₀ betains or sultains. Preferably, the carboxylate may be selected from C₂₈-alkyl-PO₂₅-EO₄₅-carboxylate.

The sulfate may be selected from at least one of TSP PO₂₋₅₀ EO₂₋₁₀₀ sulfates or other mono-, di-, and trialkylphenolalkoxysulfates, C₁₂₋₃₂-alkyl PO₂₋₅₀ EO₂₋₁₀₀ sulfates, C₁₂₋₃₂-alkyl PO₂₋₅₀ sulfates, or C₁₂₋₃₂-alkyl EO₂₋₁₀₀ sulfates. Preferably, the sulfate may be selected from TSP-PO₃₅-EO₂₀ sulfate.

The sulfonate may be selected from at least one C₁₀₋₂₀ alkylbenzene sulfonate (ABS), olefin sulfonate such as C₁₂₋₃₀ internal olefin sulfonates, C₁₂₋₂₀ alpha-olefin sulfonates, C₁₂₋₂₈ glycerol sulfonates, C₁₂₋₂₈ diphenyloxidedisulfonate.

Preferably, the sulfonate may be selected from dodecylbenzenesulfonate.

In one embodiment, the anionic co-surfactant has ammonium as counterion.

In a further preferred embodiment, the co-surfactant is a sulfate having ammonium as counterion, most preferably TSP-PO₃₅-EO₂₅-SO₄⁻ NH₄⁺.

Examples of cationic co-surfactants are ammonium salts comprising four hydrocarbon radicals, e.g. amine alkoxylate, such as (2-hydroxyethyl) (2-hydroxyhexadecyl)dimethylammonium salt as a chloride or a bromide salt. Examples of nonionic co-surfactants are alkoxylates such as polyglycol ethers, fatty alcohol polyglycol ethers, alkylphenol polyglycol ethers, end-group sealed polyglycol ethers, mixed ethers and hydroxy mixed ethers, and fatty acid polyglycol esters. Also usable are ethylene oxide/propylene oxide block polymers, fatty acid alkanolamides and fatty acid polyglycol ethers. Another important class of nonionic surfactants used according to the invention may be the polyol surfactants and here especially the glycogate surfactants, such as alkyl polyglycosides and fatty acid glucamides.

Preferred alkyl polyglycosides are in particular alkyl polyglucosides having a long-chain fatty alcohol or a mixture of long-chain fatty alcohols with branched or unbranched C₈₋₁₈ alkyl chains and the degree of oligomerization (DP) is the sugar between 1 and 10, preferably 1 to 6, in particular 1.1 to 3, most preferably 1.1 to 1.7, is, for example C₈₋₁₀ alkyl-1.5-glucoside (DP of 1.5).

Preferred fatty alcohol alkoxylates (fatty alcohol polyglycol ethers) are in particular ethylene oxide (EO) and/or propylene oxide (PO) alkoxylated, unbranched or branched, saturated or unsaturated C₈₋₂₂ alcohols having a degree of alkoxylation up to 30, preferably ethoxylated C₁₂₋₂₂ fatty alcohols having a degree of ethoxylation from less than 30, preferably 12 to 28, in particular 20 to 28, more preferably 25, for example C₁₆₋₁₈ fatty alcohol ethoxylates with 25 EO.

### EXAMPLES

The Applicant has carried out the following inventive and comparative examples to quantitatively demonstrate at least some of the advantages of the inventive process, in particular, that the undesired side reactions are minimized and that the excess of the neutralizing agent necessary is reduced.

In the examples, ammonium salt of TSP-PO₃₅-EO₂₀ sulfate is used as co-surfactant, C₁₅₋₁₆ alkyl benzene sulfonic acid is used as precursor of the ionic surfactant and NaOH is used as neutralizing agent.

One of the undesired reactions that may take place between these components is the reaction between the ammonium salt of TSP-PO₃₅-EO₂₀ sulfate with the NaOH, liberating ammonia.

To evaluate the occurrence of this undesired side reaction, the ammonium ion was measured in the co-surfactant as starting material and in the obtained chemical formulation. Ideally, the amount of ammonium cation would remain unchanged. However, if the co-surfactant would react with the neutralizing agent, the amount of ammonium cation would decrease as a result of the liberated ammonia.

### Process for Quantifying the Amount of Ammonium Cation

The content of ammonium cation was measured by ion chromatography with the 940 Professional IC Vario.

The samples were prepared as following: approximately 0.1 g and 0.2 g of the sample were weighed into 25 mL volumetric flasks, filled up to volume with the eluent and homogenized by ultrasonic treatment. For analysis by ion chromatography, the sample solutions were diluted with the eluent with regards to the calibration range. Eluent used: 2.5 mmol/L HNO₃

For quantification, an external calibration was performed using ammonium standard solutions with following concentrations: 0.1, 0.2, 0.5, 0.7, 1, 2, 5, 10 mg/L. For evaluation, the peak area was used.

### Inventive Example 1

The blending process was carried out according to a setup as illustrated in FIG. 2, wherein (1) is the source of precursor of the ionic surfactant, (2) is the source of co-surfactant and (3) is the source of neutralization agent. The ammonium salt of TSP-PO₃₅-EO₂₀ sulfate was dosed into the static mixer setup at a dosing rate of 12 g/min. The C₁₀₋₃₂ alkyl benzene sulfonic acid was dosed at a rate of 8.59 g/min. The sodium hydroxide solution was dosed at a rate of 1.915 g/min.

Per minute, a chemical formulation having the following components was prepared:

| | | | |
|---|---|---|---|
| Co- | Ammonium salt of | =5.3 wt. % | 12 g |
| surfactant | TSP-PO₃₅-EO₂₀ | | |
| | sulfate (99% active) | | |
| Precursor of | C₁₀₋₃₂ alkyl benzene sulfonic | =38.2 wt. % | 8.59 g |
| the anionic | acid (ABS) (93.1% active) | | |
| surfactant | (acid number = 147 mg | | |
| | KOH/g) | | |
| Neutraliza- | 50% NaOH in deionized | =8.5 wt. % | 1.915 g |
| tion agent | water | | |

The temperature of the blend after the neutralization but before cooling rose to 60° C. and to 38° C. after the cooler. This process led to a stable and homogeneous blend with a pH value of >8 (measured as 2 wt. % solution in deionized water), without noticeable occurrence of any gaseous by-products.

Taking into account that the ABS starting material used had an acid number of 147 mg KOH/g, which equals to 105 mg NaOH/g. This indicates that in theory 1.801 g of the NaOH 50 wt. % aq. solution should had been enough for the full neutralization of the of the ABS, i.e. equimolar of the NaOH to the acid number of the ABS. In practice, 1.915 g were needed to carry out this experiment, i.e. the molar ratio of the acid number of the precursor, i.e. ABS, to neutralization agent, i.e. NaOH, was 1:1.06.

The excess of neutralization agent needed was very small. This gives a first indication that by using the present inventive process, the undesired side-reaction.

To confirm that the undesired side-reactions between the co-surfactant and the neutralizing agent, and consequently the loss of ammonium, was greatly reduced in the present process, the amount of ammonium cation was measured in the co-surfactant as a starting material and in the obtained chemical formulation. The results are summarized in the following table:

| Ammonium cation in the Ammonium salt of | =0.003459 mol |
|---|---|
| TSP-PO₃₅-EO₂₀ sulfate | |
| Excess of NaOH used | =0.01270 mol |
| Theoretical amount of ammonium cation in the obtained | =0.003459 mol |
| chemical formulation if no side-reaction takes place | |
| Actual amount of ammonium cation in the obtained | =0.003244 mol |
| chemical formulation measured | |
| Loss of ammonium cation | 6.23% |

The results clearly show that only 6.23% of the co-surfactant reacted with the neutralization agent, producing sodium salt of TSP-PO₃₅-EO₂₀ sulfate and liberating ammonia gas. That is about 90% less than in Comparative Example 1 and about 76% less than in Comparative Example 2, as will be seen later.

### Inventive Example 2

The blending process was carried out according to a setup as illustrated in FIG. 2, wherein (1) is the source of neutralization agent, (2) is the source of co-surfactant and (3) is the source of precursor of the ionic surfactant. First, the NaOH-solution was mixed with the TSP-PO₃₅-EO₂₀ sulfate, then the C₁₀₋₃₂ alkyl benzene sulfonic acid was mixed in. The ammonium salt of TSP-PO₃₅-EO₂₀ sulfate was dosed into the static mixer setup at a dosing rate of 12 g/min. The C₁₀₋₃₂ alkyl benzene sulfonic acid was dosed at a rate of 8.59 g/min. The sodium hydroxide solution was dosed at a rate of 1.915 g/min.

Per minute, a chemical formulation having the following components was prepared:

| | | | |
|---|---|---|---|
| Co- | Ammonium salt of | =5.3 wt. % | 12 g |
| surfactant | TSP-PO₃₅-EO₂₀ | | |
| | sulfate (99% active) | | |
| Precursor of | C₁₀₋₃₂ alkyl benzene sulfonic | =38.2 wt. % | 8.59 g |
| the anionic | acid (ABS) (93.1% active) | | |
| surfactant | (acid number = 147 mg | | |
| | KOH/g) | | |
| Neutraliza- | 50% NaOH in deionized | =8.5 wt. % | 1.915 g |
| tion agent | water | | |

The temperature of the blend after the neutralization but before cooling rose to 58° C. and to 32° C. after the cooler. This process led to a stable and homogeneous blend with a pH value of >8 (measured as 2 wt. % solution in deionized water), without noticeable occurrence of any gaseous by-products.

Taking into account that the ABS starting material used had an acid number of 147 mg KOH/g, which equals to 105 mg NaOH/g. This indicates that in theory 1.801 g of the NaOH 50 wt. % aq. solution should had been enough for the full neutralization of the of the ABS, i.e. equimolar of the NaOH to the acid number of the ABS. In practice, 1.915 g were needed to carry out this experiment, i.e. the molar ratio of the acid number of the precursor, i.e. ABS, to neutralization agent, i.e. NaOH, was 1:1.06.

The excess of neutralization agent needed was very small. This gives a first indication that by using the present inventive process, the undesired side-reaction.

To confirm that the undesired side-reactions between the co-surfactant and the neutralizing agent, and consequently the loss of ammonium, was greatly reduced in the present process, the amount of ammonium cation was measured in the co-surfactant as a starting material and in the obtained chemical formulation. The results are summarized in the following table:

| Ammonium cation in the Ammonium salt of | =0.003459 mol |
|---|---|
| TSP-PO₃₅-EO₂₀ sulfate | |
| Excess of NaOH used | =0.01270 mol |
| Theoretical amount of ammonium cation in the obtained | =0.003459 mol |
| chemical formulation if no side-reaction takes place | |
| Actual amount of ammonium cation in the obtained | =0.002997 mol |
| chemical formulation measured | |
| Loss of ammonium cation | 13.44% |

The results clearly show that only 13,44% of the co-surfactant reacted with the neutralization agent, producing sodium salt of TSP-PO₃₅-EO₂₀ sulfate and liberating ammonia gas. That is about 86% less than in Comparative Example 1 and about 84% less than in Comparative Example 2, as will be seen later.

### Comparative Example 1

This comparative example is carried out according to WO 2015/138175, in particular it is based on example 2 described in page 8.

For the comparative example a 250 mL four-neck-flask was used. The flask was cooled with a water bath and was equipped with two dropping funnels. It was stirred with an agitator to mix the ingredients.

A chemical formulation having the following components was prepared:

| | | | |
|---|---|---|---|
| Co- | Ammonium salt of | =53.7 wt. % | 50 g |
| surfactant | TSP-PO₃₅-EO₂₀ | | |
| | sulfate (99% active) | | |
| Precursor of | C₁₅₋₁₆ alkyl benzene sulfonic | =36.5 wt. % | 33.98 g |
| the anionic | acid (ABS) (93.8% active) (acid | | |
| surfactant | number = 154.5 mg KOH/g) | | |
| Neutralization | 50% NaOH in deionized water | =9.8 wt. % | 9.09 g |
| agent | | | |

The flask was prefilled with the NaOH aq. solution and stirred at 200 rpm. Over a period of 1 hour, the ABS and the ammonium salt of TSP-PO₃₅-EO₂₀ sulfate were added simultaneously via the two dropping funnels. The stirring speed was incrementally adjusted from 200 rpm to 800 rpm to compensate for increasing viscosity.

During the addition of the two surfactants, the exothermic reaction led to a temperature increase which was stabilized at 40° C. by cooling of the water bath. The formation of foam and solid lumps as well as solid deposits was observed. The blend was stirred at 40° C. for another 2-3 hours at 800 rpm for homogenization.

The obtained chemical formulation had a pH value higher than 8 (measured as 2 wt. % solution in deionized water). Taking into account that the ABS starting material used had an acid number of 154.5 mg KOH/g, which equals to 110 mg NaOH/g. This indicates that in theory 7.49 g of the NaOH 50 wt. % aq. solution should had been enough for the full neutralization of the of the ABS, i.e. equimolar of the NaOH to the acid number of the ABS. However, 9.09 g were needed to carry out this experiment, i.e. the molar ratio of the acid number of the precursor, i.e. ABS, to neutralization agent, i.e. NaOH, was 1:1.21.

Due to the set-up of this process, adding excess of NaOH was necessary to compensate for the undesired side-reaction of the ammonium salt of TSP-PO₃₅-EO₂₀ sulfate with the NaOH, liberating ammonia. This was confirmed by measuring the concentration of ammonium cation in the co-surfactant before the process and in the final chemical formulation. The results are summarized in the following table:

| Ammonium cation in the Ammonium salt of | =0.0133 mol |
|---|---|
| TSP-PO₃₅-EO₂₀ sulfate | |
| Excess of NaOH used | =0.0388 mol |
| Theoretical amount of ammonium cation in the obtained | =0.0133 mol |
| chemical formulation if no side-reaction takes place | |
| Actual amount of ammonium cation in the obtained | =0.0005 mol |
| chemical formulation measured | |
| Loss of ammonium cation | 96.2% |

The results clearly show that 96.2% of the co-surfactant reacted with the neutralization agent, producing a sodium salt of TSP-PO₃₅-EO₂₀ sulfate and liberating ammonia gas.

### Comparative Example 2

Same process steps as in Comparative Example 1. But using the C₁₀₋₃₂ alkyl benzene sulfonic acid as in the inventive examples 1 and 2.

A chemical formulation having the following components was prepared:

| | | | |
|---|---|---|---|
| Co- | Ammonium salt of | =53.57 wt. % | 50 g |
| surfactant | TSP-PO₃₅-EO₂₀ | | |
| | sulfate (99% active) | | |
| Precursor of | C₁₀₋₃₂ alkyl benzene sulfonic | =37.13 wt. % | 34.66 g |
| the anionic | acid (ABS) (93.1% active) (acid | | |
| surfactant | number = 147 mg KOH/g) | | |
| Neutraliza- | 50% NaOH in deionized water | =9.30 wt. % | 8.68 g |
| tion agent | | | |

Taking into account that the ABS starting material used had an acid number of 147 mg KOH/g, which equals to 105 mg NaOH/g. This indicates that in theory 7.28 g of the NaOH 50 wt. % aq. solution should had been enough for the full neutralization of the of the ABS, i.e. equimolar of the NaOH to the acid number of the ABS. However, 8.68 g were needed to carry out this experiment, i.e. the molar ratio of the acid number of the precursor, i.e. ABS, to neutralization agent, i.e. NaOH, was 1:1.19.

As in Comparative Example 1, due to the set-up of this process, adding excess of NaOH was necessary to compensate for the undesired side-reaction of the ammonium salt of TSP-PO₃₅-EO₂₀ sulfate with the NaOH, liberating ammonia. This was confirmed by measuring the concentration of ammonium cation in the co-surfactant before the process and in the final chemical formulation. The results are summarized in the following table:

| Ammonium cation in the Ammonium salt of | =0.0150 mol |
|---|---|
| TSP-PO₃₅-EO₂₀ sulfate | |
| Excess of NaOH used | =0.0155 mol |
| Theoretical amount of ammonium cation in the obtained | =0.0150 mol |
| chemical formulation if no side-reaction takes place | |
| Actual amount of ammonium cation in the obtained | =0.0026 mol |
| chemical formulation measured | |
| Loss of ammonium cation | 82.7% |

The results clearly show that 82.7% of the co-surfactant reacted with the neutralization agent, producing a sodium salt of TSP-PO₃₅-EO₂₀ sulfate and liberating ammonia gas.

Embodiments of this Invention:
1. A continuous process for the preparation of a chemical formulation comprising at least water, an ionic surfactant and a co-surfactant, wherein the ionic surfactant is prepared in situ by reacting a precursor of the ionic surfactant with a neutralization agent and the process comprises the steps of:
   i) mixing at least one precursor of the ionic surfactant, the co-surfactant and the neutralization agent by at least one mixing device selected from the group consisting of continuous stirred tank reactors, high shear mixers, mixing pumps, static mixers and mixing nozzles,
   ii) removing the obtained chemical formulation through an outlet.
2. In a preferred embodiment of the present invention, the at least one precursor of the ionic surfactant, the co-surfactant and the neutralization agent are added to the mixing device simultaneously.
3. In a preferred embodiment of the present invention, the mixing device is a continuously stirred tank reactor or a high shear mixer.
4. In a preferred embodiment of the present invention, at least two mixing devices are used.
5. In a preferred embodiment of the present invention, step i) comprises the sub-steps of:
   i-1) combining at least one precursor of the ionic surfactant with the co-surfactant,
   i-2) feeding the mixture obtained in sub-step i-1) through at least one first mixing device,
   i-3) combining the mixture obtained in sub-step i-2) with at least one neutralization agent,
   i-4) feeding the mixture obtained in sub-step i-3) through at least one second mixing device, wherein the first and second mixing device are independently selected from a group consisting of continuous stirred tank reactors, high shear mixers, mixing pumps, static mixers and mixing nozzles.
6. In a preferred embodiment of the present invention, step i) comprises the sub-steps of
   i-1) combining the neutralization agent with the co-surfactant,
   i-2) mixing the mixture obtained in step u-1),
   i-3) adding at least one precursor of the ionic surfactant, and
   i-4) mixing the mixture obtained in step i-3).
7. In a preferred embodiment of the present invention, the first mixing device and/or and the second mixing device is a static mixer.
8. In a preferred embodiment of the present invention, the time span between the point when the precursor and the co-surfactant get in contact until the point when the homogeneous chemical formulation with the neutralized precursor is obtained is less than 15 minutes, preferably less than 2 minutes, most preferably less than 1 minute.
9. In a preferred embodiment of the present invention, the time span between the point when the neutralization agent and the co-surfactant get in contact until the point when the homogeneous chemical formulation with the neutralized precursor is obtained is less than 15 minutes, preferably less than 2 minutes, most preferably less than 1 minute.
10. In a preferred embodiment of the present invention, the precursor is acid, the neutralization agent is a base, and the ionic component is anionic.
11. In a preferred embodiment of the present invention, the precursor is selected from the group comprising alkylbenzene sulphonic acid, preferably linear alkylbenzene sulphonic acids (LABS) having an alkyl chain length of C₁₀₋₃₂; primary and secondary alkyl sulphonic acids, preferably C₁₀₋₁₅ primary alkyl sulphonic acids; alkyl ether sulphonic acids; olefin sulphonic acids; alkyl xylene sulphonic acids; dialkyl sulphosuccinic acids; and fatty acid ester sulphonic acids.
12. In a preferred embodiment of the present invention, the neutralization agent is an aqueous solution comprising a base selected from the group comprising NaOH, KOH, NH₄OH, Na₂CO₃, NaHCO₃, K₂CO₃, KHCO₃, Ca(OH)₂, Mg(OH)₂, Na acetate, K acetate or NH₄ acetate, preferably NaOH.
13. In a preferred embodiment of the present invention, the co-surfactant is anionic, preferably selected from the group comprising carboxylates, sulfonates or sulfates, most preferably sulfate.
14. In a preferred embodiment of the present invention, the anionic co-surfactant has ammonium as counterion, preferably the co-surfactant is a sulfate having ammonium as counterion, most preferably TSP-PO₃₅-EO₂₅-SO₄⁺ NH₄⁺.
15. In a preferred embodiment of the present invention, the molar ratio of the acid number or basic number of the precursor to neutralization agent is in the range of 1:1 to 1:1.5, preferably 1:1 to 1:1.1.
16. In a preferred embodiment of the present invention, the surfactant active content of the obtained chemical formulation is at least 80 wt. %, preferably 85 wt. %, based on the total weight of all components in the chemical formulation.
17. In a preferred embodiment of the present invention, the coefficient of variation of the concentration of the mixture prepared in sub-step i-2) is less than 10%, preferably less than 5%, more preferably less than 1%.
18. In a preferred embodiment of the present invention, the residence time of the components inside the mixing device is less than 1 minute, preferably less than 30 seconds, more preferably less than 15 seconds.

1.-13. (canceled)

14. A continuous process for the preparation of a chemical formulation comprising at least water, an ionic surfactant and a co-surfactant, wherein the ionic surfactant is prepared in situ by reacting a precursor of the ionic surfactant with a neutralization agent and the process comprises the steps of:
i) mixing at least one precursor of the ionic surfactant, the co-surfactant and the neutralization agent by at least one mixing device selected from the group consisting of continuous stirred tank reactors, high shear mixers, mixing pumps, inline mixers, static mixers and mixing nozzles,
ii) removing the obtained chemical formulation through an outlet, wherein
the precursor of the ionic surfactant is an acid,
the co-surfactant anionic having ammonium as counterion, and
the neutralization agent is a base not comprising an ammonium cation, and
wherein step i) comprises the sub-steps of:
i-1) combining at least one precursor of the ionic surfactant with the co-surfactant,
i-2) mixing the mixture obtained in step i-1),
i-3) adding at least one neutralization agent, and
i-4) mixing the mixture obtained in step i-3).

15. The process according to claim 14, wherein at least two mixing devices are used, wherein the mixing devices are independently selected from continuous stirred tank reactors, high shear mixers, mixing pumps, static mixers, inline mixers and mixing nozzles.

16. The process according to claim 14, wherein the mixing step i-2) is carried out in a first mixing device and the mixing step i-4) is carried out in a second mixing device,
wherein the first and second mixing device are independently selected from a group consisting of continuous stirred tank reactors, high shear mixers, mixing pumps, static mixers, inline mixers and mixing nozzles.

17. The process according to claim 15, wherein the first mixing device or and the second mixing device is a static mixer.

18. The process according to claim 14, wherein the time span between the point when the precursor and the co-surfactant get in contact until the point when the homogeneous chemical formulation with the neutralized precursor is obtained is less than 15 minutes or wherein the time span between the point when the neutralization agent and the co-surfactant get in contact until the point when the homogeneous chemical formulation with the neutralized precursor is obtained is less than 15 minutes.

19. The process according to claim 14, wherein the precursor is selected from the group consisting of alkylbenzene sulphonic acid; primary and secondary alkyl sulphonic acids; alkyl ether sulphonic acids; olefin sulphonic acids; alkyl xylene sulphonic acids; dialkyl sulphosuccinic acids; and fatty acid ester sulphonic acids.

20. The process according to claim 19, wherein the precursor is selected from the group consisting of linear alkylbenzene sulphonic acids (LABS) having an alkyl chain length of C₁₀₋₃₂ and C₁₀₋₁₅ primary alkyl sulphonic acids.

21. The process according to claim 14, wherein the neutralization agent is an aqueous solution comprising a base selected from the group consisting of NaOH, KOH, Na₂CO₃, NaHCO₃, K₂CO₃, KHCO₃, Ca(OH)₂, Mg(OH)₂, Na acetate and K acetate.

22. The process according to claim 14, wherein the co-surfactant is selected from the group consisting of carboxylates, sulfonates and sulfates.

23. The process according to claim 14, wherein the co-surfactant is a sulfate having ammonium as counterion.

24. The process according to claim 23, wherein the sulfate having ammonium as counterion is TSP-PO₃₅-EO₂₅-SO₄⁻ NH₄⁺.

25. The process according to claim 14, wherein the molar ratio of the acid number of the precursor to neutralization agent is in the range of 1:1 to 1:1.5.

26. The process according to claim 14, wherein the surfactant active content of the obtained chemical formulation is at least 80 wt. %, based on the total weight of all components in the chemical formulation.

27. The process according to claim 14, wherein the coefficient of variation of the concentration of the mixture prepared in sub-step i-2) is less than 10%,
wherein the coefficient of variation of the concentration (CV) is calculated by the formula
$$CV = \frac{s}{\overline{x}} \times 100 \%$$
where s is the concentration of a randomized sample of the mixture and x̅ is the average concentration of the mixture.

28. The process according to claim 14, wherein the residence time of the components inside the mixing device is less than 1 minute,

29. The process according to claim 28, wherein the residence time of the components inside the mixing device is less than 30 seconds.
